(19) United States
(12) Patent Application Publication
(10) Pub. No.: US 2024/0079149 A1
(43) Pub. Date: Mar. 7, 2024

(54) SYSTEMS AND METHODS FOR DESIGNING DRUG COMBINATION THERAPIES

(71) Applicant: REGENTS OF THE UNIVERSITY OF MICHIGAN, Ann Arbor, MI (US)

(72) Inventor: Sriram Chandrasekaran, Ann Arbor, MI (US)

(21) Appl. No.: 18/139,943

(22) Filed: Apr. 26, 2023

Related U.S. Application Data
(60) Provisional application No. 63/335,620, filed on Apr. 27, 2022.

(51) Int. Cl. G16H 70/40; G06N 20/00

(52) U.S. Cl. CPC G16H 70/40; G06N 20/00

(57) ABSTRACT

Systems, methods, and computer-readable medium storing instructions of using transfer machine learning for predicting drug interaction outcomes include: obtaining a trained machine learning model, obtaining genetic information of pathogens of interest, generating predicted drug interaction outcome data for drug treatments of interest using the machine learning model, and indicating the predicted drug interaction outcome data. The machine learning model may be trained by obtaining training data, classifying the training data into subsets corresponding to different actual outcomes, and generating the machine learning model using the classified subsets. The training data may include drug interaction outcome data having, for each respective pathogen of the pathogens, an outcome of drug treatments applied to the respective pathogen. The predicted drug interaction outcome data may be generated based on the genetic information of the pathogens of interest or genetic information or clinical information of living subjects having the pathogens of interest.

### FIELD OF THE DISCLOSURE

The present disclosure generally relates to using transfer learning techniques of machine learning (e.g., a random forest model) to predict drug interaction outcomes for pathogens.

### BACKGROUND

Drug resistance is a global health crisis as the pace of current antibiotic drug development is challenged by growing resistance of pathogens to a large number of available drugs. Every year tens of millions of people will contract infections from drug-resistant pathogens, and many will die from these infections. There are various pathogens which have developed drug resistance and may pose major threats to humanity. For example, according to a 2019 CDC report, carbapenem-resistant Acinetobacter is an urgent threat and multidrug-resistant Pseudomonas aeruginosa and drug-resistant tuberculosis are serious threats. However, no new classes of drugs have been developed into treatments for decades.

One possible solution to the growing problem of drug-resistant pathogens is drug combination therapy which may involve combining already-existing existing drugs to form drug combination therapies. Drug combination therapies may engage multiple cellular targets, which is difficult to achieve with a single synthetic compound and is critical to suppressing drug resistance.

While drug combination therapies may be an effective approach to combating drug resistance, the inherent combinatorial nature of drug combination therapy and the vast number of already-existing drugs makes finding new and effective drug combination therapies difficult. For example, for each possible combination of drugs, all drug-to-drug interactions must be considered to avoid negative interactions (it is frequently desired in drug combination therapy development to have synergistic interaction between drugs as opposed to antagonistic interaction). As a result of the immense combinatorial candidate space for drug combination therapies, advancement of new drug combination therapies has been slow. For example, a combination regimen of four drugs and six months of treatment for M. tuberculosis has not changed in 50 years, which has in turn led to a growing resistance of the pathogen to the combination regimen.

The combinatorial nature of drug combination therapy is not the only challenge facing development of new and effective drug combination therapies. Another challenge lies with the pathogens themselves. Generating drug interaction data for pathogens is expensive and time consuming, and accordingly, not all pathogens have drug interaction data, let alone statistically-significant sample sizes of drug interaction data. As a result, conventional techniques of developing a new and effective drug combination therapy for a certain pathogen, which relies on using drug interaction data for the pathogen, may be hindered if corresponding drug interaction data for the certain pathogen is sparse or non-existent. Accordingly, for at least the reasons above, improved techniques are needed to develop new and effective drug combination therapies for combatting the impending global health crisis of drug-resistant pathogens.

### BRIEF SUMMARY

The present application discloses a method, system, and computer-readable medium storing instructions for of using transfer machine learning for predicting drug interaction outcomes for pathogens, including: (a) obtaining a machine learning model for predicting drug interaction outcomes for pathogens, the machine learning model trained using drug interaction outcome data for a plurality of pathogens, wherein the drug interaction outcome data includes, for each respective pathogen of the plurality of pathogens, an outcome of one or more drug treatments applied to the respective pathogen; (b) obtaining genetic information of a pathogen of interest; (c) generating, using the machine learning model, predicted drug interaction outcome data for one or more drug treatments of interest applied to the pathogen of interest, based on the genetic information of the pathogen of interest; and (d) indicating the predicted drug interaction outcome data for the one or more drug treatments of interest applied to the pathogen of interest. In some aspects, the machine learning model may use a transfer learning model by changing underlying weights or structure of the machine learning model based on information from the pathogen of interest.

The term “pathogen” is used broadly herein to refer to prokaryotic and eukaryotic microorganisms including viruses, fungi, and protozoan pathogens which may be found in living organisms such as animals (e.g., humans, livestock, etc.) or plants (e.g., agriculture, fungi, algae, etc.). While the term “pathogen” is used herein, it should also be understood, in some aspects, the term “pathogen” may include non-pathogenic model organisms. The term “drug” is broadly used herein to refer to any synthetic chemical compounds, natural products or biologic molecules which may be used for purposes including treating infections caused by pathogens.

In some aspects, the drug interaction outcome data further includes genetic information or clinical information of each of a plurality of living subjects and each of the plurality of living subjects has at least one of the plurality of pathogens. Such aspects may further include: (a) obtaining genetic information or clinical information of a living subject of interest having the pathogen of interest; and wherein (b) generating, using the machine learning model, the predicted drug interaction outcome data for one or more drug treatments of interest applied to the pathogen of interest, is further based on the genetic information or clinical information of the living subject of interest. The term “living subject” is broadly used herein to refer to any living organism that may contract a pathogen, such as animals (e.g., humans, livestock, etc.) or plants (e.g., agriculture, fungi, algae, etc.).

Some aspects may include, obtaining drug information for the plurality of individual drugs of interest, wherein each of the one or more drug treatments of interest includes two or more individual drugs of interest of the plurality of individual drugs of interest.

Still further aspects may include: (a) identifying a recommended drug treatment out of the one or more drug treatments of interest based on the predicted drug interaction outcome data for each of the one or more drug treatments of interest; and (b) indicating the recommended drug treatment.

Some aspects may include: (a) analyzing feedback from a user following the indication of the predicted drug interaction outcome data for the one or more drug treatments of interest applied to the pathogen of interest, the feedback regarding the predicted drug interaction outcome data and including user input; and (b) updating, by the one or more processors, the machine learning model based on the feedback from the user.

In some aspects, a statistical model may be trained by a method including: (a) obtaining a set of training data for a plurality of pathogens including actual outcomes of one or more drug treatments applied to each of the plurality of pathogens; (b) classifying the set of training data into a plurality of subsets each corresponding to a different actual outcome or a range of actual outcomes; and (c) generating the statistical model for predicting an outcome of applying a drug treatment of interest to a pathogen of interest using the classified subsets of training data. In some aspects, the statistical model may be the machine learning model as in any of the previous aspects.

### BRIEF DESCRIPTION OF THE DRAWINGS

The skilled artisan will understand that the figures described herein are included for purposes of illustration and are not limiting on the present disclosure. The drawings are not necessarily to scale, emphasis instead being placed upon illustrating the principles of the present disclosure. It is to be understood that, in some instances, various aspects of the described implementations may be shown exaggerated or enlarged to facilitate an understanding of the described implementations. In the drawings, like primary characters throughout the various drawings generally refer to functionally similar or structurally similar components.

FIG. 1 is a simplified block diagram of an exemplary system for training or using a model to predict drug interaction outcomes for pathogens.

FIG. 2 depicts an exemplary process for training or using a multispecies transfer learning model to predict drug interaction outcomes for pathogens.

FIG. 3A-3F depict exemplary experimental data comparing drug interaction outcome prediction performance of present techniques of a multispecies transfer learning model to an exemplary single-species model.

FIG. 4 is a flow diagram depicting an exemplary method for predicting drug interaction outcomes for pathogens based on genetic information of the pathogens.

FIG. 5 is a flow diagram depicting an exemplary method for predicting drug interaction outcomes for pathogens based on genetic information of the pathogens and genetic information or clinical information of living subjects having the pathogens.

FIG. 6 is a flow diagram depicting an exemplary method for recommending a drug treatment.

FIG. 7 is a flow diagram depicting an exemplary method for training a statistical model to predict drug interaction outcomes for pathogens.

### DETAILED DESCRIPTION

The present disclosure aims to reduce problems with conventional techniques (e.g., as described in the Background section) by providing techniques for using transfer machine learning for predicting drug interaction outcomes for pathogens. The present techniques may include training or obtaining a model that generates the predicted drug interaction outcome data for the pathogens.

Present techniques offer a high-throughput approach which reduces the challenges inherent to the combinatorial nature of drug combination therapy so that researchers may efficiently screen and prioritize promising drug combinations based on individual drug data. Furthermore, present techniques of transfer learning allow for learning patterns from abundant data from modeled and well-researched pathogens (e.g., model organisms) which may then be applied to numerous clinical pathogens that have only limited corresponding data. Accordingly, present techniques will allow for more accurate identification of novel, synergistic drug combinations across various pathogens. The present techniques outperform a single-species control model in predicting drug interaction outcomes for pathogens.

Advantageously, present techniques to improve predicting drug interaction outcomes for pathogens may alter which drugs are used, an order of drugs used, or a timing of how drugs are administered to a patient. Accordingly, present techniques may improve patient care and outcome over convention techniques.

Also advantageously, present techniques to improve predicting drug interaction outcomes for pathogens may improve efficiency of drug clinical trials. In improving predictions for drug interaction outcomes, researchers may be more able to identify drug trials that are less likely to have desired outcomes (and can accordingly end the corresponding drug trial sooner, thereby reducing resources used in the drug trial) and identify new drug trials that are more likely to have desired outcomes.

Also advantageously, present techniques to improve predicting drug interaction outcomes for pathogens may be used to combat fast evolution of drug-resistant pathogens as possible drug treatments may be more quickly identified for new or evolving pathogens. Conventional methods may be ineffective at combatting this challenge as pathogens, may in some cases, evolve faster than researchers can identify possible drug treatments.

Additional advantages of present techniques over conventional approaches characterizing a recipe of a production system for producing a product will be appreciated throughout this disclosure by one having ordinary skill in the art. The various concepts and techniques introduced above and discussed in greater detail below may be implemented in any of numerous ways, and the described concepts are not limited to any particular manner of implementation. Examples of implementations are provided below for illustrative purposes.

### Exemplary System for Training and Operating a Multispecies Transfer Learning Model

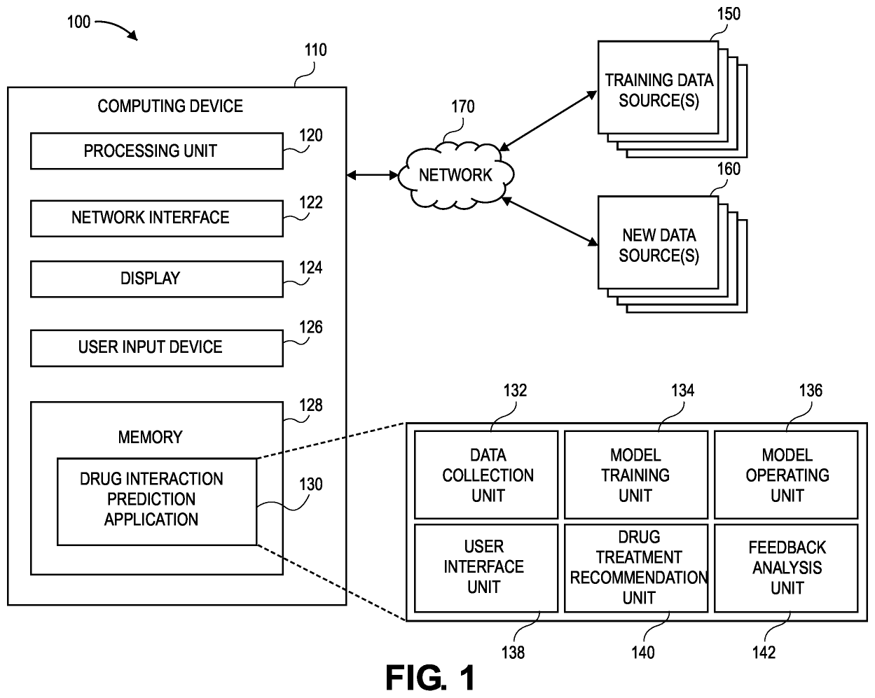

FIG. 1 is a simplified block diagram of an exemplary system 100 for training or using a model to predict drug interaction outcomes for pathogens. In some aspects, the system 100 may include standalone equipment, though in other examples the system 100 may be incorporated into other equipment. At a high level, the system 100 includes a client computing device 110, one or more training data sources 150, and one or more new data sources 160, at least some of which may be communicatively coupled via a network 170 that may be a proprietary network, a secure public internet, a virtual private network, or some other type of network, such as dedicated access lines, plain ordinary telephone lines, satellite links, cellular data networks, combinations of these, etc. Where the network 170 comprises the Internet, data communications may take place over the network 170 via an Internet communication protocol. In some aspects, more instances of the various components of the system 100 may be included (e.g., one instance of the computing device 110, twenty instances of the training data sources 150, two instances of the new data sources 160, etc.) may be included in the system 100. In some aspects, fewer instances of the various components of the system 100 may be included, for example, if the system 100 is to be used only for training a model to predict drug interaction outcomes for pathogens, the new data sources 160 may not be included; whereas, if the system 100 is used only for using a trained model to predict drug interaction outcomes for pathogens, the training data sources 150 may not be included.

As previously discussed, the computing device 110 may be included in the system 100. The computing device 110 may include a single computing device, or multiple computing devices that are either co-located or remote from each other. The computing device 110 may be generally configured to train or use a model to predict drug interaction outcomes for pathogens.

In aspects where the computing device 110 may be configured to train a statistical model to predict drug interaction outcomes for pathogens, the computing device 110 may be generally configured to: (a) obtain a set of training data for a plurality of pathogens including actual outcomes of one or more drug treatments applied to each of the plurality of pathogens; (b) classify the set of training data into a plurality of subsets each corresponding to a different actual outcome or a range of actual outcomes; and (c) generate the statistical model for predicting an outcome of applying a drug treatment of interest to a pathogen of interest using the classified subsets of training data.

In aspects where the computing device 110 may be configured to use a machine learning model to predict drug interaction outcomes for pathogens, the computing device 110 may be generally configured to: (a) obtain the machine learning model for predicting drug interaction outcomes for pathogens, the machine learning model trained using drug interaction outcome data for a plurality of pathogens, wherein the drug interaction outcome data includes, for each respective pathogen of the plurality of pathogens, an outcome of one or more drug treatments applied to the respective pathogen; (b) obtain genetic information of a pathogen of interest; (c) generate, using the machine learning model, predicted drug interaction outcome data for one or more drug treatments of interest applied to the pathogen of interest, based on the genetic information of the pathogen of interest; and (d) indicate the predicted drug interaction outcome data for the one or more drug treatments of interest applied to the pathogen of interest.

Components of the computing device 110 may be interconnected via an address/data bus or other means. The components included in the computing device 110 may include a processing unit 120, a network interface 122, a display 124, a user input device 126, and a memory 128, discussed in further detail below.

As noted above, the example system 100 includes one or more training data sources 150 and one or more new data sources 160. Each of the sources 150 and 160 may be a single source or include multiple sources that are either co-located or remote from each other. The sources 150 or 160 may provide information to the computing device 110 via the network 170. The provided information may be data, such as nominal data, ordinal data, discrete data, or continuous data. The provided information may be in the form of a suitable data structure, which may be stored in a suitable format, such as of one or more of: JSON, XML, CSV, etc. The sources 150 or 160 may provide information to the computing device 110 automatically, or in response to a request. For example, a user of the computing device 110 may wish to train a model for predicting drug treatment outcomes for pathogens. In response, the training data source 150 may send information to the computing device 110 via the network 170. The sources 150 or 160 may be databases of information themselves or may be configured to receive information, such as via user input or from other external sources (e.g., server databases). The sources 150 or 160 may be publicly-accessible; for example, the training data sources 150 may include data/information from publicly-accessible research studies or literature.

The training data sources 150 generally include training data that may be used when training a model to predict drug treatment outcomes for pathogens. The training data may include drug interaction outcome data. The drug interaction outcome data may include an outcome of one or more drug treatments applied to a respective pathogen of a plurality of pathogens. Each of the one or more drug treatments may be comprised of one or more individual drugs. The drug interaction outcome data may include information regarding properties of one or more pathogens, one or more drugs, or one or more living subjects for training. Pathogen information included in the drug interaction outcome data may include for each respective pathogens of the one or more pathogens, transcriptomics data, chemogenomics data, or gene orthology data. Drug information included in the drug interaction outcome data may include for each respective drug of the one or more drugs, transcriptomics data, chemogenomics data, chemical structures data, prior use data, or measures of synergistic/neutral/antagonistic interaction with other drugs. Living subject information included in the drug interaction outcome data may include for each respective living subject of the one or more living subjects, genetic information, demographic information, clinical information, patient information, family history, or medical records. The drug interaction outcome data may be labeled. Labels may include a measure of synergism or antagonism of a respective drug treatment applied to a respective pathogen. Labels may include a measure of efficacy of a respective drug treatment applied to a respective pathogen. To illustrate one example of drug interaction outcome data in an exemplary system for training a model to predict drug interaction outcomes for pathogens, the drug interaction outcome data may include pathogens Mycobacterium tuberculosis (M. tb) H37Rv and Escherichia coli (E. coli) K12. The drug properties may include transcriptomics, chemogenomics, chemical structures data, prior drug use data, or measures of synergistic/neutral/antagonistic interaction with other drugs.

The new data sources 160 generally include new data that may be used when using a model to predict drug treatment outcomes for pathogens. The new data may include information about one or more pathogens of interest, one or more drugs/drug treatments of interest, or one or more living subjects of interest. For example, if researchers are interested in exploring possible drug treatments for a pathogen of interest, the new data may include pathogen information such as genetic information (e.g., chemogenomics data, transcriptomics data, gene orthology data, etc.) associated with the pathogen of interest. In another example, if researchers are interested in treating a living subject of interest having a pathogen, the new data may include living subject information (e.g., genetic information, demographic information, clinical information, patient information family history, medical records, etc.) associated with the living subject of interest. In yet another example, if researchers are interested in exploring possible uses of a drug/drug treatment of interest, the new data may include drug information (e.g., chemogenomics data, chemical composition data, prior drug use data, or measures of synergistic/neutral/antagonistic interaction with other drugs, etc.) associated with the drug/drug treatment of interest.

In some aspects, the system 100 may omit one or more of the sources 150 or 160, and instead receive data/information locally, such as via user input. Techniques for receiving data/information corresponding to the sources 150 or 160 without using the sources 150 or 160 are further described and illustrated, for example, directly at the computing device 110.

Referring again to the computing device 110, the processing unit 120 includes one or more processors, each of which may be a programmable microprocessor that executes software instructions stored in the memory 128 to execute some or all of the functions of the computing device 110 as described herein. Alternatively, one or more of the processors in the processing unit 120 may be other types of processors (e.g., application-specific integrated circuits (ASICs), field-programmable gate arrays (FPGAs), etc.).

The network interface 122 may include any suitable hardware (e.g., front-end transmitter and receiver hardware), firmware, or software configured to use one or more communication protocols to communicate with external devices or systems (e.g., the sources 150 or 160). For example, the network interface 122 may be or include an Ethernet interface. The computing device 110 may communicate with any devices that provide an interface between the computing device 110 via a single communication network, or via multiple communication networks of one or more types (e.g., one or more wired or wireless local area networks (LANs), or one or more wired or wireless wide area networks (WANs) such as the Internet or an intranet, etc.).

The display 124 may use any suitable display technology (e.g., LED, OLED, LCD, etc.) to present information to a user, and the user input device 126 may be a keyboard or other suitable input device. In some aspects, the display 124 and the user input device 126 are integrated within a single device (e.g., a touchscreen display). Generally, the display 124 and the user input device 126 may combine to enable a user to interact with graphical user interfaces (GUIs) or other (e.g., text) user interfaces provided by the computing device 110, e.g., for purposes such as displaying one or more flow profiles, displaying parameters, recommending changes to one or more parameters, notifying users of equipment faults or other deficiencies, etc.

The memory 128 includes one or more physical memory devices or units containing volatile or non-volatile memory, and may or may not include memories located in different computing devices of the computing device 110. Any suitable memory type or types may be used, such as read-only memory (ROM), solid-state drives (SSDs), hard disk drives (HDDs), etc. The memory 128 stores instructions of one or more software applications that can be executed by the processing unit 120, including a drug interaction prediction (DIP) application 130. In the example system 100, the DIP application 130 includes a data collection unit 132, a model training unit 134, a model operating unit 136, a user interface unit 138, a drug treatment recommendation unit 140, and a feedback analysis unit 142. The units 132-142 may be distinct software components or modules of the DIP application 130, or may simply represent functionality of the DIP application 130 that is not necessarily divided among different components/modules. For example, in some aspects, the data collection unit 132 and the user interface unit 138 are included in a single software module. Moreover, in some aspects, the units 132-142 are distributed among multiple copies of the DIP application 130 (e.g., executing at different components in the computing device 110), or among different types of applications stored and executed at one or more devices of the computing device 110.

The data collection unit 132 is generally configured to receive (via, e.g., sources 150 or 160, user input received via the user interface unit 138, or other suitable means) training data (e.g., drug interaction outcome data) or new data (e.g., genetic information of a pathogen of interest, genetic information or clinical information of a living subject of interest, etc.).

The model training unit 134 is generally configured to train a statistical model to predict drug interaction outcomes for pathogens by obtaining training data (obtained, e.g., via/from the user input device 126, the data collection unit 132, the training data sources 150, etc.), classifying the training data into subsets corresponding to different/range of different actual outcomes, and generating the statistical model using the classified subsets.

The model operating unit 136 is generally configured to use a machine learning model (e.g., the statistical model generated by the model training unit 134, or a suitable model obtained from elsewhere) to predict drug interaction outcomes for pathogens by obtaining the machine learning model, obtaining genetic information of a pathogen of interest, generating predicted drug interaction outcome data for one or more drug treatments of interest applied to the pathogen of interest using the machine learning model, and indicating the predicted drug interaction outcome data.

The user interface unit 138 is generally configured to receive training or new data (e.g., from the sources 150 or 160). In some aspects, the user interface unit 138 is generally configured to display outputs of a model (e.g., the machine learning model operated by the model operating unit 136).

The production system operating unit 140 is generally configured to identify and indicate (e.g., by causing an electronic display to display information) a recommended drug treatment based on predicted drug interaction outcome data (e.g., the predicted drug interaction outcome data generated by the model operating unit 136).

The feedback analysis unit 142 is generally configured to receive and analyze feedback from a user following an indication of predicted drug interaction outcome data (e.g., the predicted drug interaction outcome data generated by the model operating unit 136) generated by a model, the feedback regarding the predicted drug interaction outcome data and including user input (e.g., a user selection received via the user interface unit 138). In some aspects, the feedback analysis unit 142 is generally configured to update the model based on the feedback.

In some aspects, one or more of units 132-142 are omitted (e.g., the drug treatment recommendation unit 140 may be omitted if recommended drug treatments are determined by a healthcare provider after he or she views the predicted drug interaction outcome data for one or more drug treatments of interest). The operation of each of the units 132-142 is described in further detail below, with reference to the operation of the system 100.

### Exemplary Process for Training and Operating a Multispecies Transfer Learning Model

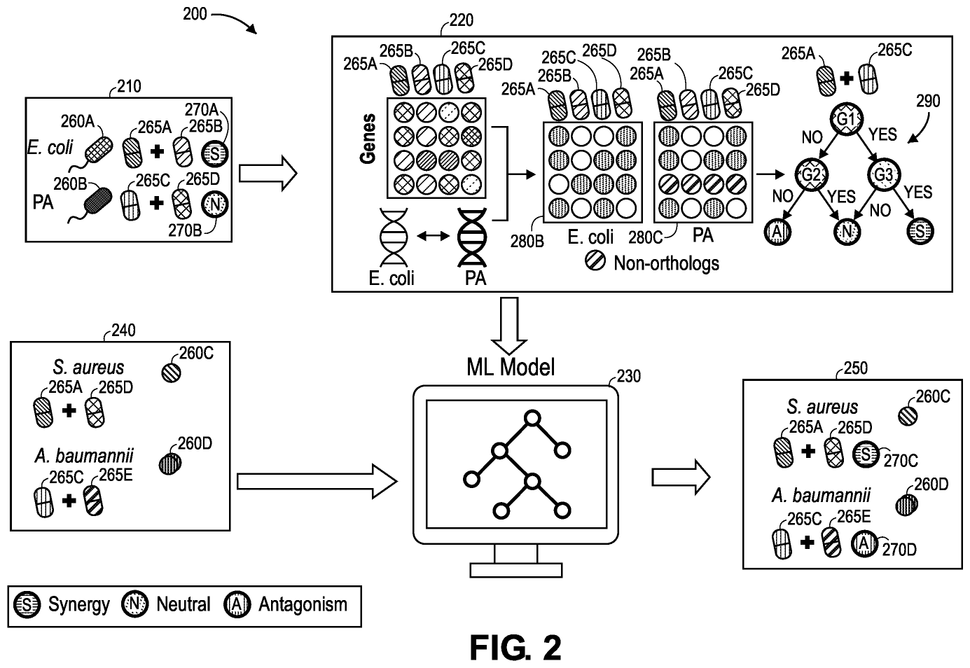

FIG. 2 depicts an exemplary process 200 for training and operating a multispecies transfer learning model to predict drug interaction outcomes for pathogens. At a high level, the process 200 includes obtaining training data 210 which includes drug interaction outcome data, training a machine learning model according to a subprocess 220 using the training data 210, obtaining the trained machine learning mode 230, obtaining new data 240 at the machine learning model 230, and indicating predicted drug interaction outcome data 250 generated by the machine learning model 230. The process 200 may use at least some of the components of the system 100 of FIG. 1. For example, the training data 210 may be received from the training data sources 150 using the data collection unit 132, the machine learning model 230 may be trained using the model generating unit 134, the machine learning model may be operated using the model operating unit 136, or prediction outputs from the machine learning model may be indicated using the user interface unit 138.

In some aspects, the process 200 may be performed in separate manners. For example, obtaining the training data 210 and training the machine learning model according to the subprocess 220 may be done at a first time by a first entity, while obtaining the trained machine learning model 230, obtaining the new data 240 at the machine learning model 230, and indicating the predicted drug interaction outcome data 250 generated by the machine learning model 230 may be done at a second time, later than the first time, by a second entity, separate from the first entity.

### Exemplary Training Data for the Multispecies Transfer Learning Model

Training the machine learning model 230 uses the drug interaction outcome data as the training data 210, as illustrated. The drug interaction outcome data may include labeled outcomes of experimental drug treatments applied to various pathogens. In addition, the drug interaction outcome data may include drug information, pathogen information, or living subject information. For example, the drug interaction outcome data may include (i) omics data characterizing drug response, which may be chemogenomics or transcriptomics data, or (ii) gene orthology data between pathogens to enable training on multiple sources of drug interaction data from various pathogens.

As illustrated, the drug interaction outcome data includes data/information associated with two pathogens (E. coli 260A and Pseudomonas aeruginosa (PA) 210B). The pathogens 260A and 260B may be useful as training data because, for example, the pathogens 260A and 260B are generally well-researched and accordingly have large amounts of corresponding outcome data. Furthermore, the pathogens 260A and 260B are known to have growing resistance to various drugs and are part of pathogen families/species that have been identified as health threats. While these are some examples of traits of pathogens that may be good candidates to include in the drug interaction outcome data used as training data, there are several other reasons why a pathogen, group of pathogens may be chosen. For example, choosing a group of pathogens, to be included in the drug interaction outcome data used as training data, that together represent a diverse set of genotypes of pathogens may be useful for training the machine learning model to make predictions about a wide variety of pathogens included in the new data.

As illustrated, the drug interaction outcome data of the training data 210 includes data/information associated with two different combinations of drugs 265A-265D applied to the two pathogens 260A and 260B. The drugs 265A-265D may represent a variety of different types of drugs or treatments which may have been applied to the pathogens 260A and 260B. While each drug treatment includes a combination of only two drugs as illustrated, it is worth noting that drug treatments of any number of drugs may be used as training data (e.g., drug treatments of only one drug, drug treatments of five drugs, drug treatments of twenty drugs, etc.). It is also worth noting that additional information about drug treatments beyond just which drugs comprise drug treatments may be included in drug interaction outcome data; for example, ratios/amounts of each drug, frequency for administering each drug, instructions for administering each drug, length of time to administer each drug, possible substitutes for each drug, shelf-life of each drug, etc. may be included as well.

As illustrated, the drug interaction outcome data of the training data 210 is labeled as having an outcome of synergy, neutral, or antagonism. The drug treatment of the drug 265A+the drug 265B applied to the E. coli pathogen 260A corresponds to label 270A of synergy. The drug treatment of the drug 265C+the drug 265D applied to the PA pathogen 260B corresponds to label 270B of neutral. Determining whether a drug combination interacts synergistically, neutrally, or antagonistically in inhibiting pathogen growth may be based on a quantified score. Synergy is when the combined effect of individual drugs is greater than the additive effect of the individual drugs, whereas antagonism is when the combined effect of the individual drugs is less than the additive effect of the individual drugs. The labels 270A and 270B may be based on scores calculated using the Bliss Independence Model, the Loewe Additivity Model or other suitable models. Each model uses different null hypotheses to define additivity. The Bliss Independence Model assumes response additivity while the Loewe Additivity Model assumes dose additivity. Additionally, the Bliss Independence Model assumes that two compounds do not interact with each other, while the Loewe Additivity Model assumes that a compound is additive or cannot interact with itself. Deviations from these null hypotheses of additivity infer synergistic or antagonistic interactions and can be graphically determined using an isobologram.

Drug interactions may alternatively be quantified by determining the fractional inhibitory concentration index (FICᵢ) using a checkerboard assay. The FICᵢ quantifies the concentrations of drugs used in combination to achieve a desired inhibitory effect. FICᵢ for any number of drugs may be calculated as follows:

$$\begin{matrix}{FIC}_{i} = {\sum }_{n = 1}^{N} {FIC}_{n} = {FIC}_{1} + {FIC}_{2} + … + {FIC}_{N} \\ = \frac{{MIC}_{1} ( in combination )}{{MIC}_{1} ( alone )} + \frac{{MIC}_{2} ( in combination )}{{MIC}_{2} ( alone )} + … + \\ \frac{{MIC}_{N} ( in combination )}{{MIC}_{N} ( alone )}\end{matrix} \qquad ( Equation 1 )$$

As shown in Equation 1, the fractional inhibitory concentration (FIC) is calculated for each drug in the combination by dividing the minimum inhibitory concentration (MIC) of the drug when in the presence of the other drugs by the MIC of the drug when alone. The FIC for each drug is then summed to get a FICᵢ representing the drug interaction score. When using FICᵢ, a FICi≤0.5 is typically deemed synergistic, while a FICi>4 is deemed antagonistic, and values in between are deemed as additive. A synergistic interaction can also be thought of as at least a four-fold decrease in the MIC of each individual drug in the pair, which produces a FICᵢ of ≤0.5.

While the outcomes included in the drug interaction outcome data of the training data 210 are illustrated as being one of synergism, neutral, or antagonism, it is worth noting any other suitable outcome labels may be used (e.g., efficacy, patient recovery/survival rate, pathogen kill rate, etc.).

In some examples, the drug interaction outcome data of the training data 210 may be obtained with outcomes pre-labeled, such as from prior research/literature/databases, etc. In other examples, the drug interaction outcome data is obtained without outcomes labeled, and the drug interaction outcome data is pre-processed to add labels based on other outcome data provided in the prior research/literature/databases using, for example, the techniques described above.

The drug interaction outcome data of the training data 210 may be obtained via receiving input, such as at the computing device 110, possibly via the network 170, using the data collection unit 132. In some aspects, the input may be user input (e.g., outcomes of drug treatments applied to pathogens) which may be received via, for example, the user input device 126 via the user interface unit 138 of the computing device 110. In some aspects, the input may be non-user input, such as input from a source, such as the training data source 150. When the input is non-user input, the network interface 122 or the data collection unit 132 of the computing device 110 may facilitate receiving the input.

While the drug interaction data of the training data 210 is illustrated as having only the two pathogens 210A and 210B, each corresponding to only one drug treatment, any number of pathogens may be included in the training data 210, with each pathogen corresponding to any number of drug treatments. A further example of a different number of pathogens and drug treatments included in exemplary training data is provided in Table 1, for example.

### Exemplary Process for Multispecies Transfer Learning Model Training

Once the training data 210 is obtained, the machine learning model 230 may be trained according to the subprocess 220 using the training data 210. At a high level, the subprocess 220 may train the machine learning model 230 as a classifier for predicting different classifications of outcomes for drug treatments applied to one or more pathogens of interest. More specifically, the subprocess 220 may train the machine learning model as a random forest classification or regression model. All or some of the subprocess 220 may use the model training unit 134 of the system 100.

The subprocess 220 may begin in some aspects with the machine learning model generating a matrix 280A including joint sensitivity and resistance profiles which correspond to each combination of the drugs 265A-265D. The matrix 280A may be generated based on the drug chemogenomics or the transcriptomics data and the gene orthology data of the pathogens 210A and 210B and drugs 265A-265D included in the training data. More specifically, the machine learning model generates the matrix 280A based on the similarities (sigma) and differences (delta) between the omics profiles of each of the drugs 265A-265D in combination. The training matrix is m drug interactions by n features in size, wherein the number of features is dependent on the omics data included in the drug interaction outcome data.

The subprocess 220 may continue in some aspects with converting the matrix 280A into binary matrices 280B and 280C corresponding to the pathogens 260A and 260B, respectively. For each set of drug interaction outcome data, the joint drug profiles are constructed using E. coli chemogenomics data, and the profiles for genes in PA are modified based on gene orthology mapping with E. coli. More specifically, omics profiles of each of the drugs 265A-265D may be transformed into binary (0/1) resistance or response profiles using Boolean operations. For chemogenomics data, the binary conversion may be done by identifying deletion strains that are significantly sensitive or resistant to a drug (i.e., the binary matrices 280B and 280C represent where gene-deletion strains were sensitive and resistant to different conditions). For transcriptomics data, the binary conversion may be done by identifying genes as being significantly downregulated or upregulated by the drug (i.e., the binary matrices 280B and 280C represent whether genes were downregulated or upregulated when applying different conditions). The binary matrices 280B and 280C may be roughly twice the number of rows (not illustrated) of that of the original matrix 280A, with the first half representing the sensitive/downregulated features, and the second half representing the resistant/upregulated features. Each column of values of the matrices 280B and 280C may correspond to a single drug representing a drug profile. For each drug combination of the drugs 265A-265D, the individual drug profiles are combined to create a joint drug profile. The summation of the drug profiles produces sigma scores, while the difference of the profiles produces delta scores. The sigma scores range from 0 to n drugs in combination, while the delta scores are either 0 or 1. Non-orthologs may also be denoted in the binary matrices 280B and 280C which may be determined using the reciprocal-best-BLAST hit (RBBH) procedure to generate ortholog predictions. Genes are then identified as orthologs if they are in the top BLAST hits of both genomes when each genome is BLASTed against the other.

The subprocess 220 may continue in some aspects with feeding the binary matrices 280B and 280C into a random forest 290 for inferring drug interaction scores in new pathogens of interest based on gene orthology. While the random forest 290 is illustrated as having only one tree, it will be appreciated that in practice, the random forest 290 may have a plurality of trees. The random forest 290 may be constructed using bootstrap aggregation (bagging), in which each tree of the random forest 290 is trained on the bootstrap sample and tested on an out-of-bag set. For each tree of the random forest 290, the out-of-bag error may be estimated. Then, for each feature in each tree, observations of each feature are randomly permuted and a model error is estimated using the out-of-bag observations containing permuted values of each feature. A mean difference and standard deviation between the model error and the out-of-bag error may be computed for all trees in the random forest 290, and an out-of-bag predictor importance estimate by permutation, or feature importance score, may be calculated by dividing the mean difference by the standard deviation. The greater the model error changes from modifying the observations of the feature, the more influential the feature is on the outcomes of the random forest 290. Accordingly, the features may be ranked based on importance scores indicating how much a given feature influences the predictions of the random forest 290. Features with importance scores reaching a cumulative fraction of 0.95 may be identified as the top features that explain 95% of the variance in predictions of the random forest 290.

### Exemplary Multispecies Transfer Learning Model

Once the random forest 290 is validated, in some aspects, the machine learning model 230 may be considered trained and ready for generating predicted drug interaction outcome data for pathogens. In some aspects, the trained machine learning model 230 may be stored by the system 100, for example, in the memory 128.

As discussed, the machine learning model 230 may comprise a transfer learning based set of machine learning models, where transfer learning comprises transferring knowledge from one model to another. Using transfer learning, a particular task (e.g., identification, classification, and/or or prediction) may be solved using full or part of an already pre-trained model (e.g., the machine learning model 230) on a different task. The transfer learning to a new pathogen of interest may be achieved by changing underlying weights or structure of the machine learning model 230 based on new data 240 (e.g., genetic information of a pathogen of interest).

Transfer learning may enable training of a base model that is universal, in the sense that it can be used as a basis for various pathogens, drug treatments, and living subjects, for example. In some examples, pre-training of the machine learning model 230 (e.g., as done in the subprocess 220) may be used to train multiple models of independent artificial neural networks, or multiple respective layers of a single artificial neural network. In some aspects, transfer learning refers to the ability of the machine learning model 230 to leverage the result (weights) of a first pre-training (e.g., the subprocess 220) to better initialize a second training, which may otherwise require a random initialization. The technique of combining the first pre-training and the second training, i.e., fine-tuning, advantageously boosts performance, in that the result of the training (e.g., training on the new data 240) performs better after pre-training than when no pre-training is performed. Model fine-tuning may be performed in some aspects. In some aspects, self-supervised learning may be performed to endow the machine learning model 230 with an understanding of the various pathogens, drug treatments, and living subjects during pre-training.

While the machine learning model 230 may be characterized as a random forest model and may be trained according to the descriptions included herein, it is worth noting there may be other models or other training techniques that could potentially be used in addition or in alternative.

For example, other machine learning models which may be used may be trained using a supervised or unsupervised machine-learning program or algorithm. The machine-learning program or algorithm may employ a neural network, which may be a convolutional neural network, a deep learning neural network, or a combined learning module or program that learns in two or more features or feature datasets in a particular areas of interest. For example, a generative adversarial neural network (GAN) may be used. The machine-learning programs or algorithms may also include natural language processing, semantic analysis, automatic reasoning, regression analysis, support vector machine (SVM) analysis, K-Nearest neighbor analysis, naïve Bayes analysis, clustering, reinforcement learning, or other machine-learning algorithms or techniques. The other machine learning models may involve identifying and recognizing patterns in training data in order to facilitate making predictions for new data. In some examples, due to processing power requirements of training machine learning models, a selected machine learning model may be trained using additional computing resources (e.g., cloud computing resources) based upon data provided by a server (not illustrated). The training data may be unlabeled, or the training data set may be labeled, such as by a human. Training of the selected machine learning model may continue until at least the selected model is validated and satisfies selection criteria to be used as a predictive model. In some examples, the selected machine learning model may be validated using a second subset of the training data set (commonly known as “test data”) to determine algorithm accuracy and robustness. Such validation may include applying the selected machine learning model to the test data to make predictions. The selected machine learning model may then be evaluated to determine whether performance is sufficient based upon comparing the predictions to known labels for the test data. Sufficiency criteria for validating the selected machine learning model may vary depending upon the size of the training data set available for training, the performance of previous iterations of machine learning models, or user-specified performance requirements.

### Exemplary New Data for the Multispecies Transfer Learning Model

As illustrated, once the machine learning model 230 is trained according to the subprocess 220 using the drug interaction outcome data of the training data 210, the trained machine learning model 230 may be used to predict outcomes for the new data 240. The new data 240 may include pathogens of interest, drugs/drug treatments of interest, or living subjects of interest. The new data may include genetic information (e.g., genotype, gene orthology) of the pathogens of interest, drug information (e.g., chemogenomics data, chemical composition data, prior drug use data, etc.) associated with the drugs/drug treatments of interest, or living subject information (e.g., genetic information, demographic information, clinical information, patient information, family history, medical records, etc.) associated with the living subjects of interest.

As illustrated, the new data 210 includes drug information of drug treatments of interest (the drug 265A+the drug 265D and the drug 265C+drug 265E) and genetic information of pathogens of interest (S. aureus 260C and A. baumannii 260D). In the new data 240, the drug treatments of the drug 265A+the drug 265D and the drug 265C+the drug 265E are applied to the pathogens of S. aureus 260C and A. baumannii 260D, respectively. While the drug 265E is not included in the drug interaction outcome data of the training data 210, as illustrated, the machine learning model 230, due to the transfer learning approach used in the training, may nonetheless be able to generate predicted drug interaction outcome data for drug treatments which include drug the 265E. Similarly, the machine learning model may be able to generate predicted drug interaction outcome data for the pathogens S. aureus 260C and A. baumannii 260D, which were also not included in the drug interaction outcome data used in the training. Also similarly (although not illustrated), the machine learning model may be able to generate predicted drug interaction outcome data for living subjects having the pathogens 260C and 260D who may be different than living subjects having the pathogens 260A and 260B included in the drug interaction outcome data of the training data 210.

The new data 240 may be obtained via receiving input, such as at the computing device 110, possibly via the network 170. In some aspects the input may be user input (e.g., data/information associated with pathogens of interest, drugs/drug treatments of interest, living subjects of interest, etc.) which may be received via, for example, the user input device 126 via the user interface unit 138 of the computing device 110. In some aspects, the input may be non-user input, such as input from a source, such as the new data source 160. When the input is non-user input, the network interface 122 or the data collection unit 132 of the computing device 110 may facilitate receiving the input.

### Exemplary Predictions for the New Data by the Multispecies Transfer Learning Model

As illustrated, once the new data 240 is obtained at the trained machine learning model 230, the predicted drug interaction outcome data 250 may be generated. The predicted drug interaction outcome data 250 for the new data 240 generated by the trained machine learning model 230, similar to the drug interaction outcomes of the training data 210, may include labels of synergism, neutrality, or antagonism, or other suitable outcome labels (e.g., efficacy, patient recovery/survival rate, pathogen kill rate, etc.). As illustrated, the machine learning model predicts that a drug treatment of the drug 265A+the drug 265D applied to the pathogen S. aureus 260C will have an outcome of synergism, denoted by label 270C. As illustrated, the machine learning model also predicts that a drug treatment of the drug 265C+the drug 265E applied to the pathogen A. baumannii 260D will have an outcome of antagonism, denoted by label 270D.

While the predicted drug interaction outcome data 250 include indicating outcomes via the labels 270C and 270D, outcomes may be indicated in any number of suitable manners such as: one or more of: charts, tables, plots, graphs, maps, diagrams, histograms, etc. More specific examples of suitable data visualization techniques may include one or more of: bar charts, pie charts, donut charts, half donut charts, multilayer pie charts, line charts, scatter plots, cone charts, pyramid charts, funnel charts, radar triangles, radar polygons, area charts, tree charts, flowcharts, tables, geographic maps, icon arrays, percentage bars, gauges, radial wheels, concentric circles, Gantt charts, circuit diagrams, timelines, Venn diagrams, histograms, mind maps, dichotomous keys, Pert charts, choropleth maps, Cartesian graphs, box and whisker plots, Hexbin plots, heat maps, pair plots, KDE charts, time series charts, correlograms, violin plots, raincloud plots, stem-and-leaf plots, bubble charts, pictogram graphs, or other suitable data visualization techniques. Genetic data/information included in the predicted drug interaction outcome data may be in any suitable formats, for example, one or more of: FASTA, FASTQ, SAM/BAM, BED, GFF/GTF, (big)WIG, VCF, or other suitable or similar sequence or annotation formats.

The ability for the machine learning model 230 to make predictions regarding outcomes for the new data 240 that may include pathogens of interest, drugs/drug treatments of interest, or living subjects of interest are not included in the training data 210 is one of the advantages to the multispecies transfer learning model 230 of the process 200.

### Experimental Performance of the Multispecies Transfer Learning Model

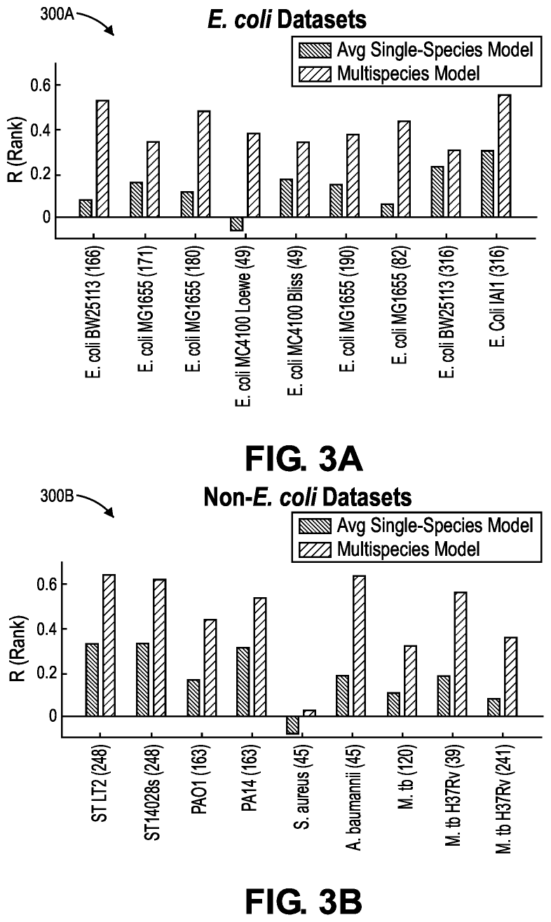

FIG. 3A-3F depict exemplary experimental data 300A-300F of performance of predicted drug interaction outcome data generated by present techniques of a multispecies transfer learning model compared to an exemplary single-species model. The multispecies transfer learning model for which the experimental performance is included may be trained or used according to a process which may be the same as or similar to the process 200 and may use at least some of the system 100.

The multispecies transfer learning model included in the experimental data 300A-300F was trained according to present techniques (e.g., the process 200 using system 100) using datasets listed in Table 1 as training data.

| | | | No. | | No. |
|---|---|---|---|---|---|
| | | | Inter- | | Unique |
| Ref. | Data Label | Pathogen Strain | actions | Scoring | Drugs |
| 1 | E. coli | E. coli | 166 | Loewe | 19 |
| | BW25113 | K-12 | | | |
| | (166) | BW25113 | | | |
| 2 | E. coli | E. coli | 171 | Loewe | 19 |
| | MG1655 | MG1655 | | | |
| | (171) | | | | |
| 3 | E. coli | E. coli | 180 | Loewe | 21 |
| | MG1655 | MG1655 | | | |
| | (180) | | | | |
| 4 | E. coli | E. coli | 49 | Loewe | 10 |
| | MC4100 | MC4100 | | | |
| | Loewe (49) | | | | |
| 5 | E. coli | E. coli | 49 | Bliss | 10 |
| | MC4100 | MC4100 | | | |
| | Bliss (49) | | | | |
| 6 | E. coli | E. coli | 190 | Bliss | 20 |
| | MG1655 | MG1655 | | | |
| | (190) | | | | |
| 7 | E. coli | E. coli | 82 | Bliss | 10 |
| | MG1655 | MG1655 | | | |
| | (82) | | | | |
| 8 | E. coli | E. coli | 316 | Bliss | 45 |
| | BW25113 | K-12 | | | |
| | (316) | BW25113 | | | |
| 9 | E. coli | E. coli | 316 | Bliss | 45 |
| | IAI1 (316) | O8 IAI1 | | | |
| 10 | ST LT2 | S. Typhimurium | 248 | Bliss | 42 |
| | (248) | LT2 | | | |
| 11 | ST14028s | S. Typhimurium | 248 | Bliss | 42 |
| | (248) | 14028s | | | |
| 12 | PAO1 | P. aeruginosa | 163 | Bliss | 42 |
| | (163) | PAO1 | | | |
| 13 | PA14 | P. aeruginosa | 163 | Bliss | 42 |
| | (163) | PA14 | | | |
| 14 | S. aureus | S. aureus | 45 | Loewe | 10 |
| | (45) | ATCC 29213 | | | |
| 15 | A. baumannii | A. baumannii | 45 | Loewe | 9 |
| | (45) | Bouvet and | | | |
| | | Grimont ATCC | | | |
| | | 17978 | | | |
| 16 | M. tb (120)* | M. tuberculosis | 120 | Loewe | 16 |
| 17 | M. tb | M. tuberculosis | 39 | Loewe | 22 |
| | H37Rv (39) | H37Rv | | | |
| 18 | M. tb | M. tuberculosis | 241 | Loewe | 52 |
| | H37Rv (241) | H37Rv | | | |

Table 1 summarizes datasets of exemplary drug interaction outcome data for pathogens E. coli, PA, ST, S. aureus, A. baumannii, and M. tb. As shown in Table 1, numerous drug interactions of unique drugs are included for each of the pathogens. Altogether, the datasets of Table 1 represent datasets of drug interactions in 11 unique pathogen strains from in various literature sources. Each of the datasets used its own unique thresholds for classifying scores as synergistic, neutral or antagonistic, but typically scores less than −0.1 to −0.25 were classified as synergistic while scores greater than 0.1 to 0.25 were typically classified as antagonistic. Scores between −0.1 and 0.1 were typically classified as neutral. These thresholds were selected based on the studies from where the data was obtained. To illustrate, Ref. 13 of Table 1 refers to pathogen strain P. aeruginosa PA14, for which 42 different unique drugs were applied and 163 interactions each have scores (which may be used to classify each interaction as one of antagonistic, neutral, or synergistic) which were determined according to the Bliss Independence Model.

To validate the multispecies transfer learning model, the multispecies transfer learning model underwent a process of training and testing using the datasets in Table 1. Each time, one dataset was tested on, while the remaining datasets were incorporated into training data. A control single-species model was also created that was only trained on the dataset E. coli MG1655 (n=171 interactions). The control model was used as a benchmark for the multispecies transfer learning model.

As shown in the experimental data 300A-300F, the multispecies transfer learning model overall performed significantly better than the control model for both E. coli and non-E. coli datasets, demonstrating the multispecies transfer learning model's ability to make more accurate predictions for data not previously seen by the multispecies transfer learning model. Performance was measured using the Spearman's rank correlation coefficient (R) and the area under ROC curves (AUC) for synergy and antagonism classification. For every test, the correlation coefficient was noticeably higher for the multispecies transfer learning model compared to that of the control model. The average R value was 0.16 for the control model across the test sets, while the average R value was 0.44 for the multispecies transfer learning model. This trend was also largely observed for AUC (synergy) (control model average AUC=0.58, multispecies transfer learning model average AUC=0.74) and AUC (antagonism) (control model average AUC=0.58, multispecies transfer learning model average AUC=0.74).

### Exemplary Process for Predicting Drug Interaction Outcomes

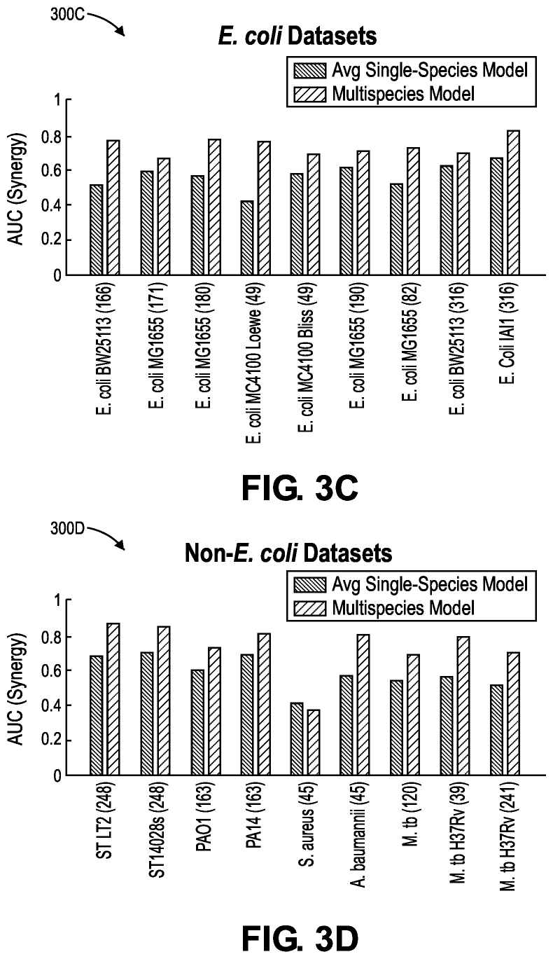

FIG. 4 is a flow diagram depicting an example method 400 for predicting drug interaction outcomes for pathogens based on genetic information of the pathogens. The method 400 may include: (i) obtaining a machine learning model for predicting drug interaction outcomes for pathogens (block 402), (ii) obtaining genetic information of a pathogen of interest (block 404), (iii) generating, using the machine learning model, predicted drug interaction outcome data for one or more drug treatments of interest applied to the pathogen of interest based on genetic information of the pathogen of interest (block 406), and (iv) indicating the predicted drug interaction outcome data for the one or more drug treatments of interest applied to the pathogen of interest (block 408). At least some of the method 400 may use the system 100 of FIG. 1. At least some of the method 400 may be the same as or similar to the process 200 illustrated in FIG. 2.

The method 400 may begin, in some aspects, with obtaining the machine learning model for predicting drug interaction outcomes for pathogens (block 402). The machine learning model may be obtained internally (e.g., by accessing files/programs/data/information stored locally in a computing system, such as the computing device 110) or externally (e.g., by receiving the machine learning model from an outside source, such as receiving the machine learning model at the computing device 110 via the network 170). The machine learning model may be fully trained once obtained, or in some aspects the machine learning model may be a template for a model to be trained. In aspects where the machine learning model has had at least some training, the machine learning model may have been trained using drug interaction outcome data for a plurality of pathogens. The drug interaction outcome data used as training data may include, for each respective pathogen of the plurality of pathogens, an outcome of one or more drug treatments applied to the respective pathogen. In addition to drug interaction outcome data for training may include one or more of chemogenomics data, transcriptomic data, or gene orthology data. The drug interaction outcome data may be labeled data used for supervised learning. Each drug treatment of the one or more drug treatments included in the drug interaction outcome data may include a at least one individual drug, and, each respective outcome of the one or more drug treatments may include an indication of a measure of synergistic interaction of the individual drugs or a measure of antagonistic interaction of the individual drugs (determined, e.g., via the Loewe Additivity model or the Bliss Independence model). While synergetic/neutral/antagonistic may be some examples of labels of respective outcomes, other suitable labels may be applied in addition or in alternative, e.g., efficacy, patient recovery/survival rate, pathogen kill rate, etc. The machine learning model may be the same as or similar to examples of a machine learning model discussed with respect to FIG. 2. For example, the machine learning model may be a classifier or regressor (e.g., a random forest model). Other examples of machine learning models which may be used may be trained using a supervised or unsupervised machine-learning program or algorithm may include a neural network (e.g., a convolutional neural network, a deep learning neural network, etc.), natural language processing, semantic analysis, automatic reasoning, regression analysis, support vector machine (SVM) analysis, K-Nearest neighbor analysis, naïve Bayes analysis, clustering, reinforcement learning, or other machine-learning algorithms or techniques.

Obtaining the genetic information of the pathogen of interest (block 404) may use one or more new data sources, such as the new data sources 160 of FIG. 1 as well as possibly the data collection unit 132 of FIG. 1. The genetic information may include genetic information chemogenomics data, transcriptometics data, gene orthology data, genotype etc. associated with the pathogen of interest. The genetic information may be provided in any suitable format used in bioinformatics/genetic research, for example, one or more of: FASTA, FASTQ, SAM/BAM, BED, GFF/GTF, (big)WIG, VCF, or other suitable or similar sequence or annotation formats. The pathogen of interest may or may not be included in the drug interaction outcome data used as training data. In aspects where the pathogen of interest is not included in the drug interaction outcome data, the pathogen of interest may have some similarities (e.g., similarities in genotype) to one or more of the pathogen included in the drug interaction outcome data and the genetic information of the pathogen of interest may be obtained, for example, by user input, external sources (e.g., the new data sources 160), or other suitable means.

Generating, using the machine learning model, the predicted drug interaction outcomes for the one or more drug treatments of interest applied to the pathogen of interest based on the genetic information of the pathogen of interest (block 406) may use a computing device, such as the computing device 110 of FIG. 1. Specifically, within the computing device an application, such as the DIP application 130, the model operating unit 136 may be used. As previously discussed, the machine learning model may be the same as or similar to (or may be representable in the same as or similar manner to) the machine learning model 230 of the process 200. The machine learning model may be used to generate the predicted drug interaction outcomes for the drug treatments of interest based on the genetic information of the pathogen of interest. In some aspects, the one or more drug treatments of interest may include at least one of the drug treatments included in the drug interaction outcome data. In some aspects, the one or more drug treatments of interest may include at least one new drug treatment not included in the drug interaction outcome data. Drug information of the at least one new drug treatment may be obtained, for example, by user input, external sources (e.g., the new data sources 160), or other suitable means.

In some aspects, the method 400 may end with indicating the predicted drug interaction outcomes (block 408). In some aspects the predicted drug interaction outcomes themselves may be displayed, while in other aspects a representation of the predicted drug interaction outcomes may be displayed. Indicating the predicted drug interaction outcomes may use a computing device, such as the computing device 110 (e.g., specifically using the display 124 or the user interface unit 138). In some aspects the predicted drug interaction outcomes itself may be stored, while in other aspects a representation of the predicted drug interaction outcomes may be stored. Storing the predicted drug interaction outcomes may use a computing device, such as the computing device 110 (e.g., specifically using the memory 128).

In some aspects, the method 400 may be performed either entirely by automation, e.g., by one or more processors (e.g., a CPU or GPU) that execute instructions stored on one or more non-transitory, computer-readable storage media (e.g., a volatile memory or a non-volatile memory, a read-only memory, a random-access memory, a flash memory, an electronic erasable program read-only memory, or one or more other types of memory), or in-part by automation and in-part by manual processes (e.g., via a human operator).

### Exemplary Process for Predicting Drug Interaction Outcomes

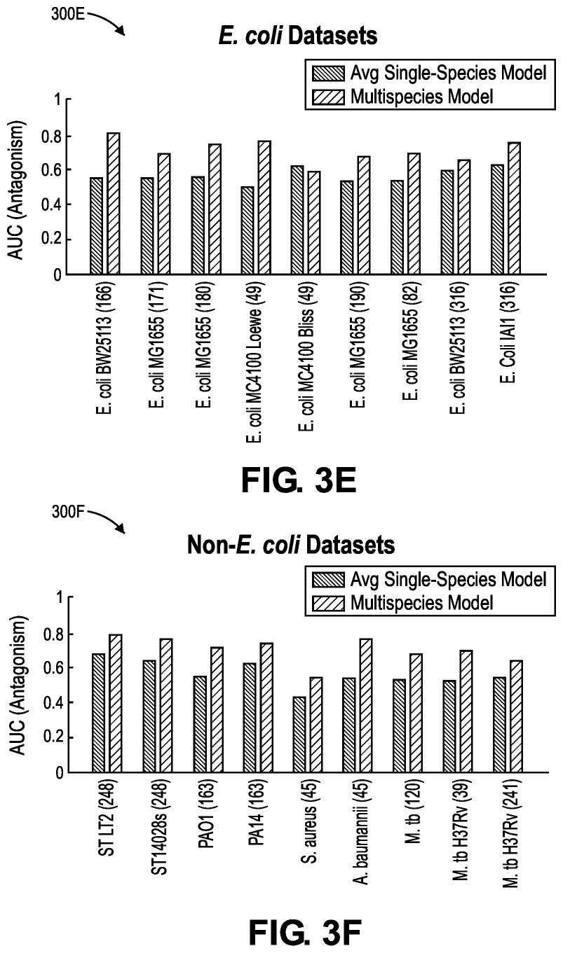

FIG. 5 is a flow diagram depicting an example method 500 for predicting drug interaction outcomes for living subjects having pathogens based on genetic information of the pathogens and genetic information or clinical information of the living subjects. The method 500 may include: (i) obtaining a machine learning model for predicting drug interaction outcomes for pathogens (block 502), (ii) obtaining genetic information of a pathogen of interest and genetic information or clinical information of a living subject of interest having the pathogen of interest (block 504), (iii) generating, using the machine learning model, predicted drug interaction outcome data for one or more drug treatments of interest applied to the pathogen of interest based on genetic information of the pathogen of interest and the genetic information or the clinical information of the living subject of interest (block 506), and (iv) indicating the predicted drug interaction outcome data for the one or more drug treatments of interest applied to the pathogen of interest (block 508). At least some of the method 500 may use the system 100 of FIG. 1. At least some of the method 500 may be the same as or similar to the process 200 illustrated in FIG. 2.

Block 502, block 504, block 506, and block 508 may each be similar to (or, in some aspects, equivalent to) each of block 402, block 404, block 406, and block 408 respectively. Certain differences between each of block 502, block 504, and block 506, with respect to each of block 402, block 404, and block 406, according to some aspects, are described herein.

In some aspects, the machine learning model obtained at block 502 may be trained using the drug interaction outcome data including the genetic information of the plurality of pathogens and the genetic information or the clinical information of the plurality of living subjects (e.g., patients, test subjects, etc. which may be human or animal) having the plurality of pathogens. In some aspects, in addition to the genetic information or the clinical information of the living subjects being included in the drug interaction outcome data, patient information, demographic information, family history, medical records, etc. associated with the living subjects may also be included.

In some aspects, the genetic information of the pathogen of interest and the genetic information or the clinical information of the subject of interest having the pathogen of interest may be obtained at block 504. The living subject of interest may or may not have been included in the plurality of living subjects in the drug interaction outcome data. In aspects where the living subject of interest is not included in the drug interaction outcome data, the living subject of interest may have some similarities (e.g., genetic similarities) to one or more of the living subjects included in the drug interaction outcome data and the genetic information or the clinical information of the living subject of interest may be obtained, for example, by user input, external sources (e.g., the new data sources 160), or other suitable means.

In some aspects, the predicted drug interaction outcomes for the one or more drug treatments of interest applied to the pathogen of interest may be generated using the machine learning model based on the genetic information of the pathogen of interest and the genetic information or the clinical information of the living subject of interest at block 506. As previously discussed, the machine learning model may be the same as or similar to (or may be representable in the same as or similar manner to) the machine learning model 230 of the process 200.

In some aspects, the method 500 may be performed either entirely by automation, e.g., by one or more processors (e.g., a CPU or GPU) that execute instructions stored on one or more non-transitory, computer-readable storage media (e.g., a volatile memory or a non-volatile memory, a read-only memory, a random-access memory, a flash memory, an electronic erasable program read-only memory, or one or more other types of memory), or in-part by automation and in-part by manual processes (e.g., via a human operator).

### Exemplary Process for Recommending Drug Treatment

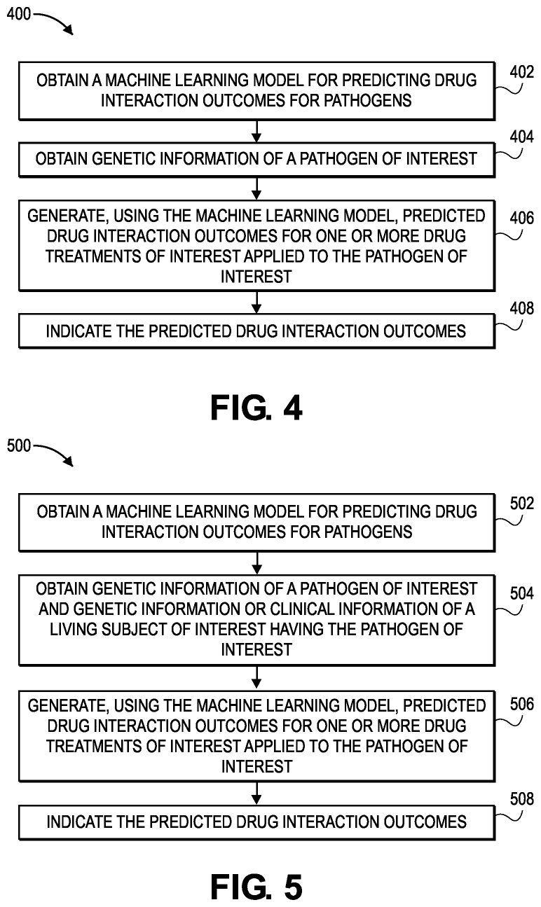

FIG. 6 is a flow diagram depicting an example method 600 for recommending a drug treatment. The method 600 may include: (i) obtaining a machine learning model for predicting drug interaction outcomes for pathogens (block 602), (ii) obtaining genetic information of a pathogen of interest (block 604), (iii) generating, using the machine learning model, predicted drug interaction outcome data for one or more drug treatments of interest applied to the pathogen of interest based on genetic information of the pathogen of interest (block 606), (iv) identifying a recommended drug treatment out of the one or more drug treatments of interest (block 608), and (v) indicating the recommended drug treatment (block 610). At least some of the method 600 may use the system 100 of FIG. 1. At least some of the method 600 may be the same as or similar to the process 200 illustrated in FIG. 2.

Block 602, block 604, and block 606 may each be similar to (or, in some aspects, equivalent to) each of block 402, block 404, block 406, respectively. In some aspects, block 602, block 604, and block 606 may each be similar to (or, in some aspects, equivalent to) each of block 502, block 504, block 506, respectively. While not illustrated in FIG. 6, in some embodiments, a block that is similar to or equivalent to block 408 or 508 may be included in the method 600.

Identifying, using the machine learning model, the recommended drug treatment out of the one or more drug treatments of interest based on the predicted drug interaction outcome data for each of the one or more drug treatments (block 608) may use a computing device, such as the computing device 110. Specifically, within the computing device an application, such as the DIP application 130, the drug treatment recommendation unit 140 may be used. The machine learning model, which may be the same as or similar to (or may be representable in the same as or similar manner to) the machine learning model 230 of the process 200, may be used to identify the recommended drug treatment based on the predicted drug interaction outcome data. For example, the machine learning model may identify the recommended drug treatment as the drug treatment of the one or more drug treatments of interest most aligned with a positive or desired outcome (e.g., most likely to kill the pathogen of interest, most likely to result in the best recovery for a living subject of interest having the pathogen of interest, most likely to have a synergetic interaction, etc.). In some aspects, the desired outcome (or priorities) may be defined according to input (e.g., the desired outcome and priority may be inputted as being to completely eradicate the pathogen of interest from a living subject of interest with less priority placed on side-effects of the drug treatment on the living subject of interest), which may be, for example, user input. In some aspects, if there is no drug treatment of the one or more drug treatments of interest that are predicted to satisfy the desired outcome, there may be no recommended drug treatment identified. In some aspects, there may be multiple drug treatments of the one or more drug treatments of interest which satisfy a desired outcome, in which case there may be multiple recommended drug treatments that are identified.

In some aspects, the method 600 may end with indicating the recommended drug treatment (block 610). In some aspects, indicating the recommended drug treatment may be the same as or similar to the indicating of the predicted drug interaction outcomes at block 408. Indicating the recommended drug treatment may use a computing device, such as the computing device 110 (e.g., specifically using the display 124 or the user interface unit 138). In some aspects the recommended drug treatment itself may be stored, while in other aspects a representation of the recommended drug treatment may be stored. Storing the recommended drug treatment may use a computing device, such as the computing device 110 (e.g., specifically using the memory 128).

In some aspects, the method 600 may be performed either entirely by automation, e.g., by one or more processors (e.g., a CPU or GPU) that execute instructions stored on one or more non-transitory, computer-readable storage media (e.g., a volatile memory or a non-volatile memory, a read-only memory, a random-access memory, a flash memory, an electronic erasable program read-only memory, or one or more other types of memory), or in-part by automation and in-part by manual processes (e.g., via a human operator).

### Exemplary Process for Training a Multispecies Machine Learning Model

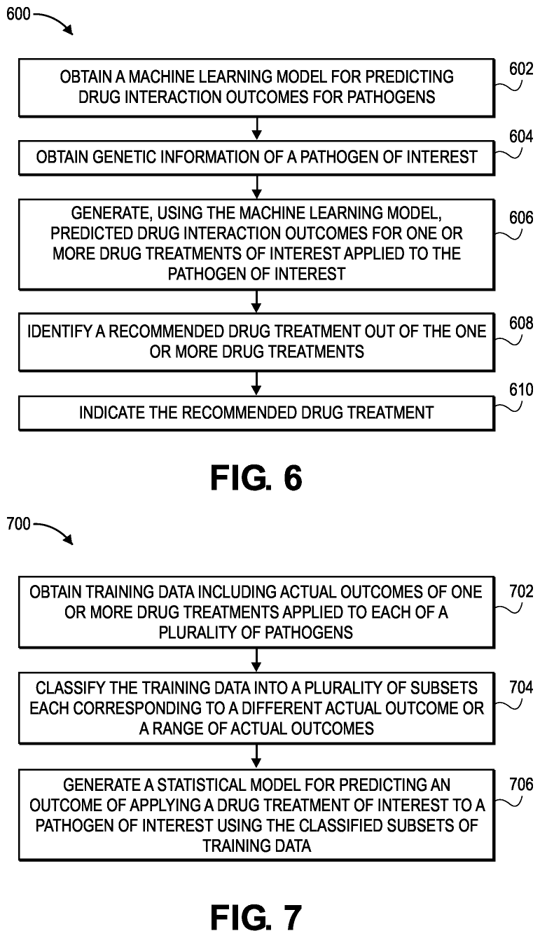

FIG. 7 is a flow diagram depicting an example method 700 for training a statistical model to predict drug interaction outcomes for pathogens. The method 700 may include: (i) obtaining training data including actual outcomes of one or more drug treatments applied to each of a plurality of pathogens (block 702), (ii) classifying the training data into a plurality of subsets each corresponding to a different actual outcome or a range of actual outcomes (block 704), and (iii) generate a statistical model for predicting an outcome of applying a drug treatment of interest to a pathogen of interest using the classified subsets of training data (block 706. At least some of the method 700 may use the system 100 of FIG. 1. At least some of the method 700 may be the same as or similar to the process 200 illustrated in FIG. 2.

The method 700 may begin, in some aspects, with obtaining the training data including the actual outcomes of the one or more drug treatments applied to each of the plurality of pathogens (block 702). In general, the training data may be the same as or similar to the drug interaction outcome data of FIGS. 1-6. More specifically, the drug interaction outcome data used as the training data may include, for each respective pathogen of the plurality of pathogens, an outcome of one or more drug treatments applied to the respective pathogen. The drug interaction outcome data may include one or more of chemogenomics data, transcriptomic data, or gene orthology data. The drug interaction outcome data may be labeled data used for supervised learning. Each drug treatment of the one or more drug treatments included in the drug interaction outcome data may include a at least one individual drug, and, each respective outcome of the one or more drug treatments may include an indication of a measure of synergistic interaction of the individual drugs or a measure of antagonistic interaction of the individual drugs (determined, e.g., via the Loewe Additivity model or the Bliss Independence model). While synergetic/neutral/antagonistic may be some examples of labels of respective outcomes, other suitable labels may be applied in addition or in alternative, e.g., efficacy, patient recovery/survival rate, pathogen kill rate, etc. The training data may be obtained internally (e.g., by accessing files/programs/data/information stored locally in a computing system, such as the computing device 110) or externally (e.g., by receiving the training data from an outside source, such as receiving the training data at the computing device 110 from the training data sources 150 via the network 170, or receiving the training data by user input using the user interface unit 138 of the system 100, etc.).

Classifying the training data into the plurality of the subsets each corresponding to a different actual outcome or a range of actual outcomes (block 406) may use a computing device, such as the computing device 110 of FIG. 1. Specifically, within the computing device an application, such as the DIP application 130, the model training unit 134 may be used. In some aspects, the model training unit 134 may classify the training data into the subsets based on labels (e.g., measures of synergistic interaction of the individual drugs, measures of antagonistic interaction of the individual drugs, efficacy, patient recovery/survival rate, pathogen kill rate, etc.). For example, drug interaction outcomes with synergetic outcomes may be classified in a first subset of the plurality of subsets, drug interaction outcomes with neutral outcomes may be classified in a second subset of the plurality of subsets, and drug interaction outcomes with antagonistic outcomes may be classified in a third subset of the plurality of subsets.

In some aspects, the method 700 may end with generating the statistical model for predicting an outcome of applying a drug treatment of interest to a pathogen of interest using the classified subsets of the training data (block 706). In some aspects, the statistical model may not be a machine learning model. In other aspects, the statistical model may be a machine learning model which may be the same as or similar to the machine learning models of FIGS. 1-6. Examples of the statistical model which include a supervised or unsupervised machine-learning program or algorithm may include a neural network (e.g., a convolutional neural network, a deep learning neural network, etc.), natural language processing, semantic analysis, automatic reasoning, regression analysis, support vector machine (SVM) analysis, K-Nearest neighbor analysis, naïve Bayes analysis, clustering, reinforcement learning, or other machine-learning algorithms or techniques. For example, the statistical model may be a classifier (e.g., a random forest model). In aspects where the statistical model is a random forest model, the model training unit 134 may collect several representative samples of each of the subsets of the training data. Using each representative sample, the model training unit 134 may generate a decision tree for determining an outcome of a drug interaction. The model training unit 134 may then aggregate or combine each of the decision trees to generate the statistical model, by for example averaging the outcomes of the drug interactions included in each individual tree, calculating a weighted average, taking a majority vote, etc. In some aspects, the model training unit 134 may also generate decision trees when the machine learning technique is boosting. In some aspects, when the statistical model is a random forest model the statistical model may be trained using a process similar to the subprocess 220 of FIG. 2. The statistical model may be stored using a computing device, such as the computing device 110 (e.g., specifically using the memory 128).

In some aspects, the method 700 may be performed either entirely by automation, e.g., by one or more processors (e.g., a CPU or GPU) that execute instructions stored on one or more non-transitory, computer-readable storage media (e.g., a volatile memory or a non-volatile memory, a read-only memory, a random-access memory, a flash memory, an electronic erasable program read-only memory, or one or more other types of memory), or in-part by automation and in-part by manual processes (e.g., via a human operator).

### Additional Considerations

Some of the figures described herein illustrate example block diagrams having one or more functional components. It will be understood that such block diagrams are for illustrative purposes and the devices described and shown may have additional, fewer, or alternate components than those illustrated. Additionally, in various aspects, the components (as well as the functionality provided by the respective components) may be associated with or otherwise integrated as part of any suitable components.

Some aspects of the disclosure relate to a non-transitory computer-readable storage medium having instructions/computer-readable storage medium thereon for performing various computer-implemented operations. The term “instructions/computer-readable storage medium” is used herein to include any medium that is capable of storing or encoding a sequence of instructions or computer codes for performing the operations, methodologies, and techniques described herein. The media and computer code may be those specially designed and constructed for the purposes of the aspects of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as ASICs, programmable logic devices (“PLDs”), and ROM and RAM devices.

Examples of computer code include machine code, such as produced by a compiler, and files containing higher-level code that are executed by a computer using an interpreter or a compiler. For example, an aspect of the disclosure may be implemented using Python, R, MATLAB, Julia, SAS, Java, C++, or other object-oriented programming language and development tools. Additional examples of computer code include encrypted code and compressed code. Moreover, an aspect of the disclosure may be downloaded as a computer program product, which may be transferred from a remote computer (e.g., a server computer) to a requesting computer (e.g., a client computer or a different server computer) via a transmission channel. Another aspect of the disclosure may be implemented in hardwired circuitry in place of, or in combination with, machine-executable software instructions.

The use herein of “a” or “an” are employed to describe elements and components of the embodiments herein. This is done merely for convenience and to give a general sense of the description. This description, and the claims that follow, should be read to include one or at least one and the singular also includes the plural unless expressly stated or it is obvious that it is meant otherwise. As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the terms “approximately,” “substantially,” “substantial,” “roughly” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the techniques disclosed herein have been described with primary to particular operations performed in a particular order, it will be understood that these operations may be combined, sub-divided, or re-ordered to form an equivalent technique without departing from the teachings of the present disclosure. Accordingly, unless specifically indicated herein, the order and grouping of the operations are not limitations of the present disclosure.

1. A computer-implemented method of using transfer machine learning for predicting drug interaction outcomes for pathogens, comprising:
obtaining, by one or more processors, a machine learning model for predicting drug interaction outcomes for pathogens, the machine learning model trained using drug interaction outcome data for a plurality of pathogens, wherein the drug interaction outcome data includes, for each respective pathogen of the plurality of pathogens, an outcome of one or more drug treatments applied to the respective pathogen;
obtaining, by the one or more processors, genetic information of a pathogen of interest;
generating, by the one or more processors using the machine learning model, predicted drug interaction outcome data for one or more drug treatments of interest applied to the pathogen of interest, based on the genetic information of the pathogen of interest; and
indicating, by the one or more processors, the predicted drug interaction outcome data for the one or more drug treatments of interest applied to the pathogen of interest.

2. The computer-implemented method of claim 1, wherein the drug interaction outcome data further includes one or both of genetic information or clinical information of each of a plurality of living subjects and each of the plurality of living subjects has at least one of the plurality of pathogens.

3. The computer-implemented method of claim 2, further comprising:
obtaining, by the one or more processors, one or both of genetic information or clinical information of a living subject of interest having the pathogen of interest; and wherein
generating, by the one or more processors using the machine learning model, the predicted drug interaction outcome data for the one or more drug treatments of interest applied to the pathogen of interest, is further based on one or both of the genetic information or the clinical information of the living subject of interest.

4. The computer-implemented method of claim 1, wherein one or both of:
(i) each drug treatment of the one or more drug treatments included in the drug interaction outcome data includes a plurality of individual drugs, and, each respective outcome of the one or more drug treatments includes an indication of a measure of synergistic interaction of the plurality of individual drugs or a measure of antagonistic interaction of the plurality of individual drugs, or
(ii) each drug treatment of interest of the one or more drug treatments of interest includes a plurality of individual drugs of interest, and, each respective outcome of the one or more drug treatments of interest includes an indication of a measure of synergistic interaction of the plurality of individual drugs of interest or a measure of antagonistic interaction of the plurality of individual drugs of interest.

5. The computer-implemented method of claim 4, wherein either the measure of synergistic interaction or the measure of antagonistic interaction, for one or both of (i) each of the individual drugs of the drug treatments, (ii) or each of the individual drugs of interest of the drug treatments of interest, includes one or more scores which are determined using one or both of the Loewe Additivity model or the Bliss Independence model.

6. The computer-implemented method of claim 1, wherein the plurality of pathogens of the drug interaction outcome data include at least two different pathogen strains, and, for each of the at least two different pathogen strains, the drug interaction outcome data includes one or more of chemogenomics data, transcriptomics data or gene orthology data.

7. The computer-implemented method of claim 1, further comprising:
obtaining, by the one or more processors, drug information for the plurality of individual drugs of interest, wherein each of the one or more drug treatments of interest includes two or more individual drugs of interest of the plurality of individual drugs of interest.

8. The computer-implemented method of claim 1, wherein one or both of:
(i) the pathogen of interest is not one of the plurality of pathogens of the drug interaction outcome data, or
(ii) at least one of the one or more drug treatments of interest is not one of the one or more drug treatments of the drug interaction outcome data.

9. The computer-implemented method of claim 1, wherein the machine learning model is a random forest model.

10. The computer-implemented method of claim 1, further comprising:
identifying, by the one or more processors, a recommended drug treatment out of the one or more drug treatments of interest based on the predicted drug interaction outcome data for each of the one or more drug treatments of interest; and
indicating, by the one or more processors, the recommended drug treatment.

11. The computer-implemented method of claim 1, further comprising:
analyzing, by the one or more processors, feedback from a user following the indication of the predicted drug interaction outcome data for the one or more drug treatments of interest applied to the pathogen of interest, the feedback regarding the predicted drug interaction outcome data and including user input; and
updating, by the one or more processors, the machine learning model based on the feedback from the user.

12. A computer system for using transfer machine learning for predicting drug interaction outcomes for pathogens, comprising:
one or more processors;
a program memory coupled to the one or more processors and storing executable instructions that, when executed by the one or more processors, cause the computer system to:
obtain a machine learning model for predicting drug interaction outcomes for pathogens, the machine learning model trained using drug interaction outcome data for a plurality of pathogens, wherein the drug interaction outcome data includes, for each respective pathogen of the plurality of pathogens, an outcome of one or more drug treatments applied to the respective pathogen;
obtain genetic information of a pathogen of interest;
generate, using the machine learning model, predicted drug interaction outcome data for one or more drug treatments of interest applied to the pathogen of interest, based on the genetic information of the pathogen of interest; and
indicate the predicted drug interaction outcome data for the one or more drug treatments of interest applied to the pathogen of interest.

13. The computer system of claim 12, wherein the drug interaction outcome data further includes one or both of genetic information or clinical information of each of a plurality of living subjects and each of the plurality of living subjects has at least one of the plurality of pathogens.

14. The computer system of claim 13, wherein the executable instructions further cause the computer system to:
obtain one or both of genetic information or clinical information of a living subject of interest having the pathogen of interest; and wherein
generating, using the machine learning model, the predicted drug interaction outcome data for the one or more drug treatments of interest applied to the pathogen of interest, is further based on one or both of the genetic information or the clinical information of the living subject of interest.

15. The computer system of any one of claim 12, wherein one or both of:
(i) each drug treatment of the one or more drug treatments included in the drug interaction outcome data includes a plurality of individual drugs, and, each respective outcome of the one or more drug treatments includes an indication of a measure of synergistic interaction of the plurality of individual drugs or a measure of antagonistic interaction of the plurality of individual drugs, or
(ii) each drug treatment of interest of the one or more drug treatments of interest includes a plurality of individual drugs of interest, and, each respective outcome of the one or more drug treatments of interest includes an indication of a measure of synergistic interaction of the plurality of individual drugs of interest or a measure of antagonistic interaction of the plurality of individual drugs of interest.

16. The computer system of any one of claim 12, wherein the plurality of pathogens of the drug interaction outcome data include at least two different pathogen strains, and, for each of the at least two different pathogen strains, the input properties data includes one or more of chemogenomics data, transcriptomics data or gene orthology data.

17. A tangible, non-transitory computer-readable medium storing executable instructions for using transfer machine learning for predicting drug interaction outcomes for pathogens, when executed by one or more processors of a computer system, cause the computer system to:
obtain a machine learning model for predicting drug interaction outcomes for pathogens, the machine learning model trained using drug interaction outcome data for a plurality of pathogens or model organisms, wherein the drug interaction outcome data includes, for each respective pathogen of the plurality of pathogens, an outcome of one or more drug treatments applied to the respective pathogen;
obtain genetic information of a pathogen of interest;
generate, using the machine learning model, predicted drug interaction outcome data for one or more drug treatments of interest applied to the pathogen of interest, based on the genetic information of the pathogen of interest; and
indicate the predicted drug interaction outcome data for the one or more drug treatments of interest applied to the pathogen of interest.

18. The tangible, non-transitory computer-readable medium of claim 17, wherein the drug interaction outcome data further includes one or both of genetic information or clinical information of each of a plurality of living subjects and each of the plurality of living subjects has at least one of the plurality of pathogens.

19. The tangible, non-transitory computer-readable medium of claim 18, wherein the executable instructions further cause the computer system to:
obtain one or both of genetic information or clinical information of a living subject of interest having the pathogen of interest; and wherein
generating, using the machine learning model, the predicted drug interaction outcome data for the one or more drug treatments of interest applied to the pathogen of interest, is further based on one or both of the genetic information or the clinical information of the living subject of interest.

20. The tangible, non-transitory computer-readable medium of claim 17, wherein one or both of:
(i) each drug treatment of the one or more drug treatments included in the drug interaction outcome data includes a plurality of individual drugs, and, each respective outcome of the one or more drug treatments includes an indication of a measure of synergistic interaction of the plurality of individual drugs or a measure of antagonistic interaction of the plurality of individual drugs, or
(ii) each drug treatment of interest of the one or more drug treatments of interest includes a plurality of individual drugs of interest, and, each respective outcome of the one or more drug treatments of interest includes an indication of a measure of synergistic interaction of the plurality of individual drugs of interest or a measure of antagonistic interaction of the plurality of individual drugs of interest.

21. A computer-implemented method for training a statistical model to predict drug interaction outcomes for pathogens, comprising:
obtaining, by one or more processors, a set of training data for a plurality of pathogens including actual outcomes of one or more drug treatments applied to each of the plurality of pathogens;
classifying, by the one or more processors, the set of training data into a plurality of subsets each corresponding to a different actual outcome or a range of actual outcomes; and
generating, by the one or more processors, the statistical model for predicting an outcome of applying a drug treatment of interest to a pathogen of interest using the classified subsets of training data.

22. The computer-implemented method of claim 21, further comprising:
generating the statistical model for predicting the outcome of applying the drug treatment of interest to the pathogen of interest using one or more machine learning techniques.

23. (canceled)

24. (canceled)
